(19) United States
(12) Patent Application Publication
(10) Pub. No.: US 2005/0026214 A1
(43) Pub. Date: Feb. 3, 2005

(54) Method of identifying glycosyl transferase binding compounds

(76) Inventors: Jacques Biton, Ouen (FR); Francois Michoux, Villiers Sur Marne (FR); Jean-Noel Veltz, Villeparisis (FR); Jacques Dumas, Martin (FR)

Correspondence Address: ROSS J. OEHLER;AVENTIS PHARMACEUTICALS INC., ROUTE 202-206, MAIL CODE: D303A, BRIDGEWATER, NJ, 08807, US

(21) Appl. No.: 10/489,034

(22) PCT Filed: Sep. 2, 2002
(86) PCT No.: PCT/FR02/02989

(30) Foreign Application Priority Data
Sep. 5, 2001 (FR) 01/11469

(51) Int. Cl. G01N 33/53

(52) U.S. Cl. 435/7.1

(57) ABSTRACT

The present invention relates to a method of identifying, screening or selecting a compound which binds to the transglycosylation site of a recombinant glycosyl transferase, comprising the steps consisting in: a) bringing said compound into contact with said recombinant protein before, after or at the same time as said recombinant protein is brought into contact with a transglycosylation activity inhibitor, said inhibitor being labeled with a label that generates a direct or indirect signal, b) studying said signal linked to said recombinant protein, the binding of said compound to the transglycosylation site being deduced from the difference between the signal obtained in step b) and the signal obtained in the absence of said compound.

The present invention relates to a method of identifying compounds which can bind to a bacterial glycosyl transferase, at the transglycosylation site. The invention also relates to the compounds obtained by such a method, and to their uses.

Peptidoglycan is a polymer synthesized by bacteria and essential to their survival. The enzymes involved in the synthesis and organization of this bacterial peptidoglycan, specific to the prokaryotic world, thus constitute very advantageous potential targets in the search for novel antibiotics.

Among these, PBPs (Penicillin Binding Proteins) which are part of the membrane steps, are the subject of many studies. This interest is related mainly to the presence of a transpeptidase activity which is inhibited by penicillins (van Heijenoort, J. 1996, p. 1025-1034. In Neidhardt et al. (ed.), Escherichia coli and Salmonella, 2^{nd} ed.: cellular and molecular biology, ASM Press, Washington, D.C.).

The most widely studied are the class A PBPs, which are modular proteins having two enzyme activities: transpeptidase activity (represented by a sequence of approximately 340 amino acids) and a glycosyl transferase activity (approximately 300 amino acids in the N-terminal region).

This glycosyl transferase activity is still relatively unknown, both due to the difficulty of following the enzyme reaction and due to a complete lack of crystallography data. An inhibitor of this activity, moenomycin, the exact mechanism of action of which remains to be elucidated, is, however, known (Wasielewski et al., 1965, Antimicrob. Agents and Chem., 743-748).

It is important to note that, to date, no class A PBP has been entirely crystallized.

Co-existing alongside these bifunctional PBPs are two monofunctional systems:
firstly, the monofunctional PBPs, which have only transpeptidase activity: one of these PBPs has recently been crystallized (Gordon et al., 2000, J. Mol. Biol., 299, 477-485).
secondly, enzymes having only glycosyl transferase activity, called MgtA (also referred to as MtgA) (Di Berardino et al., 1996, FEBS Lett., 392, 184-188).

It is important to be able to have novel glycosyl transferase activity inhibitors which could thus be used as novel antibiotics.

The present invention therefore relates to a novel method of identifying, detecting and/or screening compounds which bind to the transglycosylation site of a glycosyl transferase, this method being simple to carry out, and said glycosyl transferase being either a class A PBP protein, or a protein of MgtA type (also referred to as MtgA) , sensitive to moenomycin (for example an MgtA from staphylococcus and from streptococcus, such as S. aureus or S. pneumoniae). The method according to the invention also makes it possible to carry out a high throughput screening, i.e. makes it possible to be able to readily test several compounds at the same time. This method therefore allows a gain in time and a substantial saving to be made for the detection of novel glycosyl transferase binding partners, and an embodiment is represented diagrammatically in FIG. 1.

A certain number of methods of detecting compounds which can, inter alia, bind to the transglycosylation site of a glycosyl transferase have been published (Branstrom A., Midha S., Goldman R. FEMS Microbiool. Lett., 2000, 191, 187-190; Barbosa M., Yang G., Fang G., Kurilla M., Pompliano D, Antimicrob Agents. Chemother., 2002, 46, 4, 943-946; Vollmer W. Holtje, J. V., Antimicrob Agents. Chemother., 2000, 44, 5, 1181-1185). These methods all have a certain number of disadvantages compared to the method which is the subject of the present application. Thus, these techniques do not target specifically the transglycosylase activity in the synthesis of bacterial peptidoglycan and are not at all suitable for high throughput screening methods for discovering molecules of pharmaceutical interest.

In a first embodiment, the invention relates to a method of identifying and/or screening and/or selecting a compound which binds to the transglycosylation site of a recombinant glycosyl transferase, comprising the steps consisting in:
a) bringing said compound into contact with said recombinant protein before, after or at the same time as said recombinant protein is brought into contact with a transglycosylation activity inhibitor, said inhibitor being labeled with a label that generates a direct or indirect signal,
b) studying said signal linked to said recombinant protein,
   the binding of said compound to the transglycosylation site being deduced from the difference between the signal obtained in step b) and the signal obtained in the absence of said compound.

In a preferred embodiment, said recombinant glycosyl transferase is a class A PBP, more preferably Escherichia coli PBP1b. This protein is the main protein responsible for the glycosyl transferase activity essential to bacterial wall synthesis in vitro. Moreover, it has considerable homology with the class A PBPs observed in the other bacteria. Thus, use is preferably made of the PBP1b corresponding to SEQ ID No.2, SEQ ID No.1 representing a fusion protein constructed for the production of a recombinant PBP1b. The method can, however, be adapted using any class A PBP originating from a microorganism having a peptidoglycan, whether it is Gram +or Gram −. Use is preferably made of class A PBPs originating from microorganisms which are pathogenic for humans, for example S. aureus, S. pneumoniae, M. leprae, L. pneumophilia, M. catarrhalis, C. jeikeium, H. influenzae, P. aeruginosa, etc.

Thus, the binding to the transglycosylation site is detected by competition with the inhibitor used. This method makes it possible to obtain compounds specific for the transglycosylation activity which also exhibit a high probability of inhibiting this activity. The advantage of using a recombinant protein makes it possible to also decrease the risks of binding of the various compounds tested which may occur if a protein prepared directly from the bacterial membrane is used, the preparation thus obtained then possibly containing contaminant proteins.

In a particular embodiment, said inhibitor is moenomycin. It is, however, important to note that moenomycin analogs, such as those described in application WO 99/26956, or any other compound which inhibits transglycosylase activity, could also be used.

In a preferred embodiment, said recombinant protein is attached to a solid support. This support may in particular be a column or a flat surface. Preferably, the solid support according to the invention consists of beads bearing a group capable of attaching recombinant protein, such as copper ions or a glutathione residue. In fact, the use of beads makes it possible to bring the protein into contact with the inhibitors and the test compounds in solution, which, in general, makes it possible to improve the binding capacity, compared to a flat (two-dimensional) surface.

In a particular embodiment, said recombinant protein has been modified by genetic engineering in order to exhibit a modification allowing it to bind to said support. Such modifications are known to those skilled in the art and comprise in particular the addition of histidine residues at the N- or C-terminal end of the protein, which allows binding with a metal chelate (copper for example). Glutathione-based systems can also be used.

In one embodiment of the method according to the invention, said label is a radioactive or fluorescent label. Thus, use may be made of any type of radioactive labeling, in particular by incorporation of radioactive compounds (preferably ³H) into the structure of the inhibitor. The use of tritium is in fact preferred when the labeled inhibitor used in the method according to the invention is an organic molecule. However, the incorporation of ¹³C or ¹⁴C into the backbone of said inhibitor can also be envisioned. Thus, use may be made of a ¹⁴C-labeled precursor (glucose, propionate, etc.), during synthesis of the inhibitor, when said inhibitor is prepared by fermentation. When it is prepared by chemical synthesis, elements already labeled are used.

It is also possible to label said inhibitor with a fluorescent label or a label of another nature, whether the signal emitted is detected directly, or whether it is detected only in the event of contact (or proximity) between the protein and the inhibitor (indirect detection). Thus, both the inhibitor and the protein can be labeled with fluorescent compounds, binding between the two entities then being determined by “quenching” or by other methods (for example SPA (Scintillation Proximity Assay) or FRET (Fluorescence Resonance Energy Transfer).

Thus, in a preferred embodiment, the PBP1b is attached to SPA beads (Amersham) which contain a scintillant. These PBP-beads are brought into contact with the potential inhibitor and the labeled moenomycin. If the PBP binds the inhibitor, no signal is seen. If the PBP binds the labeled moenomycin, the proximity of the radioelement and bead triggers the emission of a signal (emission of photons by the scintillant contained in the SPA bead).

In one embodiment, the signal linked to said recombinant protein can be deduced by measuring the signal not linked to the protein, as a function of the total starting signal. In fact, since the amount of inhibitor added in the method according to the invention is known, the amount of initial signal is known. It is then possible, after the inhibitor has been passed over the protein, and the various washes have been carried out, to determine the amount of unbound inhibitor and to thus deduce therefrom the amount of inhibitor bound to the protein. This is an indirect method.

However, the embodiment in which the amount of inhibitor bound to the protein is measured directly is preferred.

The invention also relates to a method of identifying a product having antibacterial activity, comprising the steps of:
a) implementing a method according to the invention,
b) modifying the product selected in step a), in particular by grafting residues onto the chemical backbone,
c) testing the product modified in step b) in in vitro and/or in vivo methods, on relevant models for measuring antibiotic activity,
d) identifying the product which makes it possible to obtain an antibiotic activity greater than the activity obtained for the product selected in step a).

In fact, the development of a medicinal product is often carried out on the basis of the following principle:
screening of compounds having a desired activity by a suitable method,
selecting the compounds which correspond to the “specifications” (here, binding to the transglycosylation activity site of the glycosyl transferase),
determining the structure (in particular the sequence (optionally tertiary) if peptides are involved, formula and backbone if chemical compounds are involved) of the compounds selected,
optimizing the compounds selected, by modification of the structure (for example by changing the stereochemical conformation (for example changing the amino acids in a peptide from L to D)), addition of substituents to the peptide or chemical backbones, in particular by grafting residues onto the backbone, modification of peptides (see in particular Gante “Peptidomimetics”, in Angewandte Chemie-International Edition Engl. 1994, 33. 1699-1720),
passage and screening of the compounds thus obtained on suitable models which are often models closer to the pathology studied. At this stage, use is often in particular made of animal models, in general in rodents (mice, rats, etc.) or in dogs, or even primates.

The in vitro models are readily used by those skilled in the art. The compounds selected by the methods according to the invention (with optionally the structural modifications introduced) are added to a culture medium for the target bacteria, at varying concentrations, and the survival of the bacteria is studied by any suitable method, in particular by plating them out onto solid media and counting the colonies formed.

The animal models which can be used are well known to those skilled in the art. Use is, for example, made of models based on immunodepressed mice (for example scid/scid), which are infected with bacteria, which leads to the development of an infection. The effectiveness of the compounds selected by the method according to the invention is studied by resolution of the infection.

The invention also relates to a compound which can bind to the transglycosylation site of a glycosyl transferase and which preferably has antibiotic activity, which can be obtained by a method according to the invention, or directly obtained by one of said methods.

Such a compound according to the invention may be a compound having a chemical structure (of the small organic molecule type), a lipid, a sugar, a protein, a peptide, a protein-lipid, protein-sugar, peptide-lipid or peptide-sugar hybrid compound, or a protein or a peptide to which chemical branching has been added.

Among the organic compounds envisioned, they may contain one or more aromatic or nonaromatic rings, and also several residues of any kind (in particular lower alkyl, i.e. having between 1 and 6 carbon atoms) However, the compound according to the invention is not moenomycin, or the compounds of WO 99/26956.

The invention also relates to a compound according to the invention, as a medicinal product, as it is or with a pharmaceutically acceptable excipient, and also to the use of said compounds for preparing a medicinal product intended to treat bacterial infections.

The invention thus relates in particular to the use of compounds which may be or which are obtained by the method according to the invention and which have the ability to bind to the transglycosylation site of a glycosyl transferase and/or the ability to inhibit this activity, for preparing a medicinal product intended to treat bacterial infections. Preferably, the preferred compound also has antibiotic activity, which can readily be tested on animal models or in vitro, on culture media. Said compound is not moenomycin and behaves as a competitor of this product in the method according to the present invention.

An advantageous inhibitor of the glycosyl transferase site is moenomycin and, in a preferred embodiment of the invention, this compound is used after it has been tritiated, this allowing a preferred implementation of the invention, insofar as this modification does not radically modify the properties of moenomycin, and allows ready detection, in particular by SPA.

Since the number of saturated bonds has an effect on the more or less lipophilic nature of the molecule and therefore probably on the signal/background noise ratio observed during the test (increase in sticking with lipophilicity), it may be advantageous to seek to limit the number of saturated bonds.

Hydrogenation in the presence of Wilkinson's catalyst makes it possible to achieve this aim with hydrogen, but gives disappointing results with tritium.

Thus, an alternative method has been developed, consisting of tritiation in heterogeneous medium, preferably by measuring the amount of tritium absorbed and stopping the reaction at approximately two mol of tritium per mole of moenomycin. Thus, a controlled tritiation of moenomycin is carried out.

The mixture obtained is then advantageously separated by chromatography so as to group together products with identical specific activity (therefore according to the number of tritium-saturated double bonds).

Thus, the invention also relates to a method of preparing tritiated moenomycin, comprising a step of attachment of tritium to one or more double bonds of the moenomycin side chain (FIG. 2). This method is preferably carried out in heterogeneous medium, in the presence of a catalyst, in particular palladium.

Preferably, said catalyst is palladium-on-charcoal, preferably palladium at approximately 12-25% on charcoal, more preferably at approximately 18% on charcoal.

The moenomycin is preferably dissolved in an organic solvent, and preferably in ethanol or methanol. The choice of solvent suitable for moenomycin is within the scope of those skilled in the art.

In the presence of the catalyst, the reaction medium is pressurized with tritium, and brought back to a temperature of less than approximately 45-50° C., preferably less than approximately 30° C., more preferably to ambient temperature, i.e. approximately 20° C.

Stirring is carried out at ambient temperature, in order to decrease the pressure, and the reaction medium is then filtered and concentrated under vacuum, and then the residue is taken up. Stirring is thus carried out for the amount of time required for integration of one to two mol of tritium per mole of moenomycin. This period of time depends on the temperature and can be determined by those skilled in the art, but it is indicated that the time required is approximately 15 minutes when working at 20° C.

Filtration of the medium is carried out after elimination of the excess tritium.

The mixture can then be analyzed, for example by HPLC, and can be purified on a preparative column, according to protocols known to those skilled in the art.

The method according to the invention makes it possible to reproducibly obtain batches of tritiated moenomycin, and to control the amount of tritium incorporated, as a function of the amount of tritium introduced.

Thus, the method according to the invention makes it possible to obtain a tritium-labeled moenomycin such that the tritium saturates only a limited number (one or two) of double bonds, which makes it possible to conserve the characteristics and properties of the moenomycin.

In addition, separation by chromatography (HPLC) of the monosaturated and disaturated products makes it possible to work with batches having reproducible specific activity. This point may prove to be important for judging the quality of the competitor effect of the products which are tested in the method according to the invention. It is in fact advisable to have completely identified batches with well-defined properties. It should be noted that any other method of separating the products can be used.

The invention also relates to tritiated moenomycin which can optionally be or is directly obtained by the method of tritiation according to the invention, the tritium preferably being incorporated by saturation of a double bond in the side chain, and/or being synthesized by fermentation in the presence of radioactive precursors.

The invention also uses a recombinant glycosyl transferase. This membrane-bound protein is preferably prepared such that it may be solubilized, so as to be produced with a certain purity, with the aim of introducing greater specificity into the method which is the subject of the present invention. Thus, the invention relates to a method of preparing a recombinant glycosyl transferase using a vector comprising the gene of said glycosyl transferase, comprising the steps of:
a) fermentation of a cell into which said vector has been introduced, under conditions which allow the production of the recombinant glycosyl transferase;
b) purification of said recombinant glycosyl transferase in the presence of a detergent, preferably a nonionic detergent.

Preferably, this method is applied for the preparation of a class A PBP, and in particular E. coli PBP1b, the main agent responsible for the glycosyl transferase activity essential to bacterial wall synthesis in vitro.

Preferably, the vector introduced into the bacterial cell contains the PBP gene, at the end of which has been inserted a polyhistidine tail, by known molecular biology methods. This enables the binding of the recombinant protein with the SPA-type beads when the method according to the invention is implemented. A protein having a sequence similar to SEQ ID No.1, which is indicated in the illustration, is thus obtained, amino acids 1-23 corresponding to the polyhistidine tail, amino acids 24-822 corresponding to the E. coli PBP (SEQ ID No. 2).

The fermentation is carried out according to the usual methods. Depending on whether a particular promoter is used, it is possible to induce the production (or even the overproduction) of protein, which can also be effected by varying the fermentation temperature. All this is well known to those skilled in the art.

The PBP protein is a hydrophobic membrane-bound protein. It is therefore necessary to purify it in the presence of detergent. The methods of purification used are, for their part, well known to those skilled in the art. The procedure is preferably carried out in the presence of a nonionic detergent, the preferred detergent being NOG (N-octyl glucopyranoside). Another nonionic detergent may also be chosen, such as Hecameg, Triton X-100, tetraethylene glycol monooctyl ether or Nonidet P-40. It should be noted that the detergent is used in all the steps of the purification.

The method according to the invention is also preferably implemented in the presence of a nonionic detergent, and more particularly of the detergent used during the purification, i.e. NOG. This makes it possible to observe good enzyme activity.

### DESCRIPTION OF THE FIGURES

FIG. 1: diagrammatic representation of the method according to the invention. The SPA beads bearing copper groups are represented by ovals, with the site of attachment of the polyhistidine (His) end of the E. coli PBP1b protein. In the presence of an inhibitor, the labeled moenomycin ([3H]-moenomycin) cannot bind to the transglycosylation site of the protein. In the absence of the inhibitor, the binding takes place and a signal is emitted.

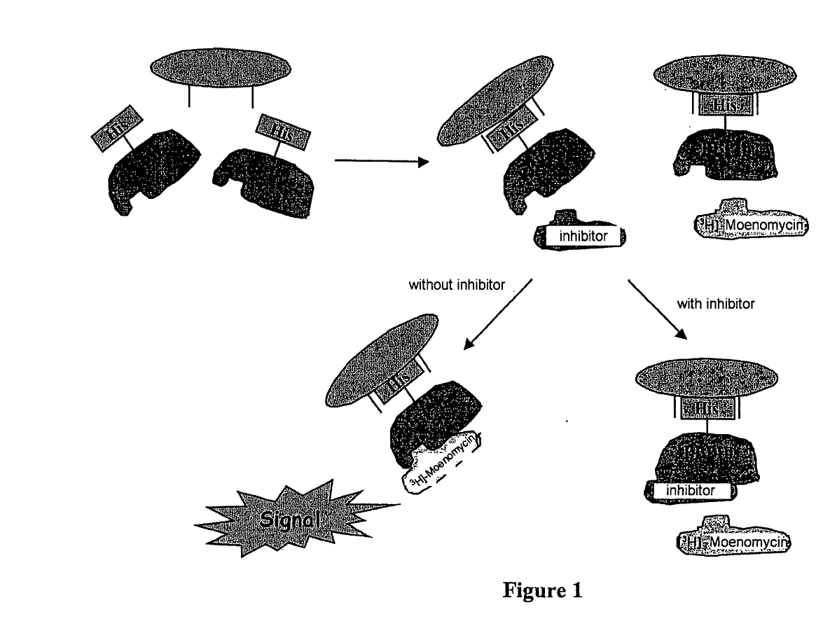

FIG. 2: representation of the tritiation of the moenomycin by saturation of a double bond on the side chain. T: tritium, Ca: catalyst.

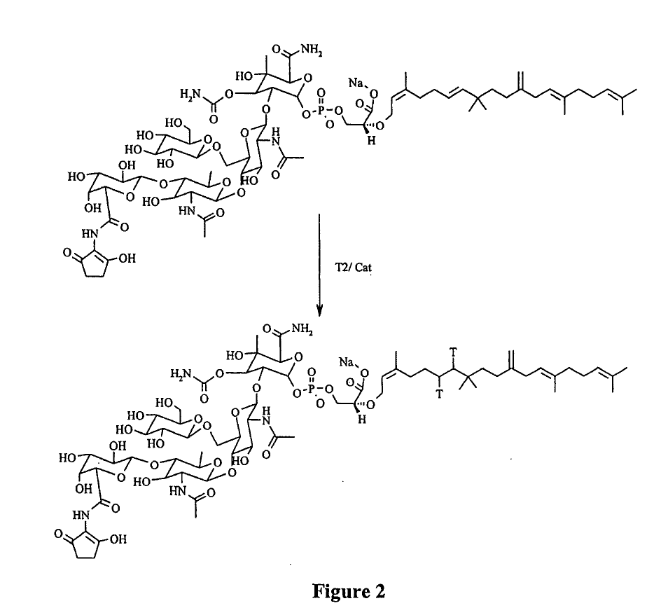

The examples below illustrate an implementation of the invention, but should not be considered as limiting the invention.

### EXAMPLES

### Example 1

### Reagents and Materials

| | |
|---|---|
| PVT copper His-Tag | 200 μg/100 μl/well |
| beads AMERSHAM: | 0.860 mg/ml; MW = 89 kDa; 8.31 μM; |
| Purified E. coli PBP1b | 5 pmol/10 μl/well |
| Moenomycin: | MW = 1580 Da; 200 pmol/10 μl/well |
| | for following the nonspecific |
| | level. |
| Inhibitor: | Dilution in DMSO; 10 μl/well |
| ³H-moenomycin: | 8.5 MBq/ml; 8.9 μM; 12.5 pmol/ |
| | 100 μl/well |
| Trizma hydrochloride | SIGMA |
| Maleic acid | MERCK |
| MgCl₂ | MERCK |
| NOG | SIGMA (n-octyl-β-D-glucopyranoside) |
| NaCl | MERCK |
| Buffer 1: | 10 mM Tris, maleate; 10 mM MgCl₂; |
| | 0.2 M NaCl; 1% NOG; pH 7.2 |
| 10x PBS | GIBCO BRL |
| Tween 20 | ACROS |
| Buffer 2: | 1x PBS; 0.5% Tween 20 |
| BSA | CALBIOCHEM |
| Buffer 3: | 2x PBS; 2% BSA |

WALLAC Microbeta 1450 radioactivity counter

### Example 2

### Protocol

2.1 Attachment of the PBP1b to the beads

After thorough stirring of the solution, the desired amount of beads is removed. The solution is diluted to ⅕in Milli-Q water. The solution is again diluted to ½in buffer 3. The solution of PBP1b is prepared by diluting buffer 1. (100 μl of beads+10 μl of PBP1b solution) times the number of wells to be prepared are mixed in a vacutainer tube. The mixture is incubated for 30 min at 37° C. at 250 rpm.

2.2 3H-Tracer/Inhibitor Competition

Preparation of the Radioinert Moenomycin for Determination of the Nonspecific Binding Level:

Stock solution at 1.58 mg/ml, i.e. 1 mM in buffer 2 (to be conserved at −80° C.). This stock solution is diluted to 1/10and then to ⅕in buffer 2.

Preparation of the ³H-moenomycin:

Stock solution at 8.9 μM. The volume necessary to have a solution at a final concentration of 125 nM is removed. Evaporation is carried out under a stream of nitrogen and the residue is taken up with the final volume of buffer 2.

Preparation of the Inhibitors:

The dilutions are prepared in DMSO. The initial concentrations are such that the inhibitor is at the correct final concentration by depositing 10 μl/well.

The following are Deposited into a Translucent Greiner 96-well Plate:

10 μl of radioinert moeno in the wells for level of nonspecific binding.
10 μl of inhibitor in the test wells.
100 μl of ³H-moeno in all the wells.
10 μl of DMSO in the wells without protein, the wells for level of maximum binding, the wells for level of nonspecific binding.

The beads/PBP1b are washed twice in 1×PBS; 0.5% Tween 20 in order to remove the unattached PBP1b.

Between each washing/suction, centrifugation is carried out for 5 min at 1000 G at ambient temperature.

After the final centrifugation, the beads/PBP1b are taken up with (110 μl of buffer 2)×number of wells to be prepared.

110 μl of beads/PBP1b are deposited per well.

The plate is covered with a self-adhesive plastic film.

The plate is incubated overnight at 4° C. without shaking.

Incubations for 24 h, 48 h and even 72 h at 4° C. do not give significantly different results.

Counting

Counting is carried out without washing or centrifugation on a radioactivity counter.

### Example 3

### Calculations

The percentage inhibition of binding of the ³H-moenomycin with each concentration of inhibitor, relative to the maximum binding (well for level of maximum binding), is calculated.

A curve for % inhibition=f ([inhibitor]) is plotted in order to determine the IC50.

### Example 4

### Summary of Concentrations

| | [initial] | pmol/well | [final] |
|---|---|---|---|
| Beads | 2 mg/ml | 200 μg/100 μl | 0.9 mg/ml |
| PBP1b | 500 nM | 5 pmol/10 μl | #24 nM |
| ³H-moenomycin | 125 nM | 12.5 pmol/100 μl | 57 nM |
| Inhibitors | ±1 mM | ±10 nmol/10 μl | ±45 μM |

### Example 5

### High Throughput Assay

The use of the method according to the invention made it possible to test approximately 500 000 compounds in 5 days, and to select approximately 1000 thereof. Thus, the method is rapid, with high throughput, and relatively discriminating.

### Example 6

### Tritiation of the Moenomycin

The following are introduced into a 1 cm³ round-bottomed tritiation flask: 6 mg of moenomycin, i.e. ˜10 μmol. 300 μl of methanol. 2 mg of palladium 18%-on-charcoal catalyst (Degussa E10N/D).

The mixture is placed on the bench, trapped, placed under vacuum, and pressurized with tritium.

After return to 20° C., the mixture is agitated for 15 min in order to obtain a decrease in pressure of 400 mbar, i.e. approximately 20 μmol of tritium (total volume of tritium 1 cm³).

After recovery of the excess tritium, the reaction medium is filtered and concentrated under vacuum, and the residue is taken up with 100 cm³ of ethanol and counted.

1.1 Ci are obtained (in theory for 20 μmol of tritium 1.2 Ci).

The Product is Analyzed by HPLC Under the Following Conditions:

C8 symmetry column, 5μ, 3.9×150 mm
Solvent: acetonitrile/water/TFA: 55/45/0.1
Flow rate: 1 cm³/min
Detection: UV 220 nm and radioactivity.

The composition of the mixture is as follows: unchanged product: 24% (by UV). Monosaturated: 29%, disaturated: 28% (remainder to 100% of radioactivity: polysaturated products).

Purification on Preparative Column:

C8 symmetry column, 7μ, 7.8×300 mm.
Solvent: acetonitrile/water/TFA: 55/45/0.1
Flow rate: 4 cm³/min.
Detection: UV 220 nm and radioactivity.

Characteristics of Two Batches:

| | | |
|---|---|---|
| Batch A: | Total activity: | 6.56 GBq (177 mCi) |
| | Specific activity: | 1.9 TBq/mmol (˜2 T/mol) |
| | Activity by volume: | 37 MBq/cm³ (ethanol |
| | | containing 5% water) |
| | Storage: | −80° C. under inert gas. |
| Batch B: | Total activity: | 8.28 GBq (223 mCi). |
| | Specific activity: | 4.92 TBq/mmol (˜4.5 T/mol) |
| | Activity by volume: | 37 MBq/cm³ (ethanol |
| | | containing 5% water) |
| | Storage: | −80° C. under inert gas. |

1-13 cancel

14. A method for identifying a compound which binds to the transglycosylation site of a recombinant glycosyl transferase, comprising the steps of:
(a) measuring the activity of a transglycosylation activity inhibitor on said recombinant glycosyl transferase in the absence of said compound, said inhibitor comprising a label that generates a direct or indirect signal; and
(b) measuring the activity of a transglycosylation activity inhibitor on said recombinant glycosyl transferase in the presence of said compound,
the binding of said compound to the transglycosylation site being deduced from the difference between the signal obtained in step (b) and the signal obtained in step (a).

15. The method of claim 14, wherein said inhibitor is moenomycin.

16. The method of claim 14, wherein said recombinant glycosyl transferase is attached to a solid support.

17. The method of claim 16, wherein said solid support comprises copper groups.

18. The method of claim 14, wherein said inhibitor comprises a label selected from the group consisting of: a radioactive label and a fluorescent label.

19. The method of claim 14, wherein said signal is measured directly.

20. The method of claim 14, wherein said signal is measured indirectly.

21. The method of claim 20, wherein said signal is measured by a Scintillation Proximity Assay.

22. The method of claim 20, wherein said signal is measured by a Fluorescence Resonance Energy Transfer.

23. The method of claim 14, wherein the signal linked to said recombinant protein is deduced by measuring the signal not linked to the protein as a function of the total starting signal.

24. The method of claim 14, wherein said inhibitor is tritiated.

25. A method of identifying a product having antibacterial activity, comprising the steps of:
(a) identifying a compound which binds to the transglycosylation site of a recombinant glycosyl transferase;
(b) measuring the antibiotic activity of the compound identified in step (a);
(c) modifying the compound identified in step (a);
(d) measuring the antibiotic activity of the compound modified in step (c),
wherein said product is determined to have an antibiotic activity if the antibiotic activity measured in step (d) is greater than the antibiotic activity measured in step (b).

26. The method of claim 25, wherein said identifying step (a) comprises the method of claim 14.

27. The method of claim 25, wherein said modifying step (b) comprises grafting residues onto the chemical backbone of said compound identified in step (a).

28. The method of claim 25, wherein said modifying step (b) comprises changing the stereochemistry of said compound identified in step (a).

29. An antibiotic comprising a product identified by the method of claim 12 and a pharmaceutically acceptable adjunct.

30. A method of preparing tritiated moenomycin, comprising the step of attaching a tritium to one or more double bonds of the moenomycin side chain.

31. A moenomycin exhibiting a molecule of tritium incorporated into its backbone.

32. A method of preparing a recombinant glycosyl transferase using a vector comprising the gene of said glycosyl transferase, comprising the steps of:
a) fermentation of a cell into which said vector has been introduced, under conditions which allow the production of the recombinant glycosyl transferase;
b) purification of said recombinant glycosyl transferase in the presence of a detergent, preferably a nonionic detergent.


SEQUENCE LISTING (2 sequences)

SEQ ID NO: 1 - protein, 822 aa, Artificial
  fusion protein for producing recombinant E. coli PBP1b
MGSSHHHHHHSSGLVPRGSHMASMPRKGKGKGKGRKPRGKRGWLWLLLKLAIVFAVLIAIYGVYLDQKIRSRIDGKVWQLAAAVYGRMVNLEPDMTISKNEMVKLLEATQYRQVSKMTRPGEFTVQANSIEMIRRPFDFPDSKEGQVRARLTFDGDHLATIVNMENNRQFGFFRLDPRLITMISSPNGEQRLFVPRSGFPDLLVDTLLATEDRHFYEHDGISLYSIGRAVLANLTAGRTVQGASTLTQQLVKNLFLSSERSYWRKANEAYMALIMDARYSKDRILELYMNEVYLGQSGDNEIRGFPLASLYYFGRPVEELSLDQQALLVGMVKGASIYNPWRNPKLALERRNLVLRLLQQQQIIDQELYDMLSARPLGVQPRGGVISPQPAFMQLVRQELQAKLGDKVKDLSGVKIFTTFDSVAQDAAEKAAVEGIPALKKQRKLSDLETAIVVVDRFSGEVRAMVGGSEPQFAGYNRAMQARRSIGSLAKPATYLTALSQPKIYRLNTWIADAPIALRQPNGQVWSPQNDDRRYSESGRVMLVDALTRSMNVPTVNLGMALGLPAVTETWIKLGVPKDQLHPVPAMLLGALNLTPIEVAQAFQTIASGGNRAPLSALRSVIAEDGKVLYQSFPQAERAVPAQAAYLTLWTMQQVVQRGTGRQLGAKYPNLHLAGKTGTTNNNVDTWFAGIDGSTVTITWVGRDNNQPTKLYGASGAMSIYQRYLANQTPTPLNLVPPEDIADMGVDYDGNFVCSGGMRILPVWTSDPQSLCQQSEMQQQPSGNPFDQSSQPQQQPQQQPAQQEQKDSDGVAGWIKDMFGSN

SEQ ID NO: 2 - protein, 799 aa, Escherichia coli
MPRKGKGKGKGRKPRGKRGWLWLLLKLAIVFAVLIAIYGVYLDQKIRSRIDGKVWQLAAAVYGRMVNLEPDMTISKNEMVKLLEATQYRQVSKMTRPGEFTVQANSIEMIRRPFDFPDSKEGQVRARLTFDGDHLATIVNMENNRQFGFFRLDPRLITMISSPNGEQRLFVPRSGFPDLLVDTLLATEDRHFYEHDGISLYSIGRAVLANLTAGRTVQGASTLTQQLVKNLFLSSERSYWRKANEAYMALIMDARYSKDRILELYMNEVYLGQSGDNEIRGFPLASLYYFGRPVEELSLDQQALLVGMVKGASIYNPWRNPKLALERRNLVLRLLQQQQIIDQELYDMLSARPLGVQPRGGVISPQPAFMQLVRQELQAKLGDKVKDLSGVKIFTTFDSVAQDAAEKAAVEGIPALKKQRKLSDLETAIVVVDRFSGEVRAMVGGSEPQFAGYNRAMQARRSIGSLAKPATYLTALSQPKIYRLNTWIADAPIALRQPNGQVWSPQNDDRRYSESGRVMLVDALTRSMNVPTVNLGMALGLPAVTETWIKLGVPKDQLHPVPAMLLGALNLTPIEVAQAFQTIASGGNRAPLSALRSVIAEDGKVLYQSFPQAERAVPAQAAYLTLWTMQQVVQRGTGRQLGAKYPNLHLAGKTGTTNNNVDTWFAGIDGSTVTITWVGRDNNQPTKLYGASGAMSIYQRYLANQTPTPLNLVPPEDIADMGVDYDGNFVCSGGMRILPVWTSDPQSLCQQSEMQQQPSGNPFDQSSQPQQQPQQQPAQQEQKDSDGVAGWIKDMFGSN